(19) United States
(12) Patent Application Publication
(10) Pub. No.: US 2013/0289333 A1
(43) Pub. Date: Oct. 31, 2013

(54) PARTICLE BEAM THERAPY SYSTEM

(71) Applicants: Kengo Sugahara, Tokyo (JP); Masahiro Ikeda, Tokyo (JP); Kazushi Hanakawa, Tokyo (JP); Taizo Honda, Tokyo (JP); Hisashi Harada, Tokyo (JP)

(72) Inventors: Kengo Sugahara, Tokyo (JP); Masahiro Ikeda, Tokyo (JP); Kazushi Hanakawa, Tokyo (JP); Taizo Honda, Tokyo (JP); Hisashi Harada, Tokyo (JP)

(73) Assignee: MITSUBISHI ELECTRIC CORPORATION, Chiyoda-ku (JP)

(21) Appl. No.: 13/977,093

(22) PCT Filed: Mar. 7, 2011
(86) PCT No.: PCT/JP2011/055236

§ 371 Date: Jun. 28, 2013

(51) Int. Cl. A61N 5/10

(52) U.S. Cl. CPC A61N 5/1077
USPC 600/1

(57) ABSTRACT

The present invention is intended to enable proper elimination of the remanent magnetization of the scanning magnet, which is used in a particle beam therapy system, in a short time. In the particle beam therapy system that irradiates an irradiation target with a particle beam 18 accelerated by an accelerator and scanned by scanning magnets 11 and 12, power supplies 13 and 14 to operate the scanning magnets 11 and 12 output pattern currents for demagnetizing the scanning magnets 11 and 12. The pattern current is controlled by a control circuit 15 that reads a demagnetization pattern 17 and controls the power supplies 13 and 14.

### TECHNICAL FIELD

The present invention relates to a particle beam therapy system that scans a charged particle beam accelerated by an accelerator with a scanning magnet and irradiates an irradiation object with the particle beam.

### BACKGROUND ART

Typically, a particle beam therapy system includes abeam generator that generates a charged particle beam, an accelerator that is connected to the beam generator and accelerates the generated charged particle beam, a beam transport system for transporting the charged particle beam emitted after being accelerated to the energy set by the accelerator, and a particle beam irradiation apparatus that is provided at the downstream side of the beam transport system and irradiates the affected area of the patient, who is an irradiation target, with the charged particle beam.

In a particle beam irradiation apparatus that is of a scanning irradiation type to form a radiation field by scanning a thin pencil-shaped beam so as to match the shape of an irradiation target, high irradiation position accuracy is required in order to prevent particle beam irradiation to normal tissue other than the affected area. In order to satisfy this requirement, it is necessary to completely eliminate the remanent magnetization of the particle beam scanning magnet. The method of eliminating the remanent magnetization of the electromagnet is disclosed in PTL 1 and PTL 2, for example.

### CITATION LIST

### Patent Literature

[PTL 1x] JP-A-63-133506
[PTL 2] JP-A-10-229014

### SUMMARY OF INVENTION

### Technical Problem

In a particle beam therapy system including two scanning magnets, which control the scanning of a supplied charged particle beam in different directions so that the supplied charged particle beam is shaped into a three-dimensional irradiation shape based on the treatment plan, and a power supply that operate the electromagnet, three-dimensional irradiation is performed on the different affected area for each patient. Therefore, assuming that a patient B is treated after a certain patient A is treated, it is necessary to completely eliminate the influence of the remanent magnetization of the scanning magnet after completing the treatment of the patient A and then to start the treatment of the patient B. If remanent magnetization remains after the treatment of the patient A, an irradiation shape planned for the patient B becomes different from the affected area of the patient B.

In addition, when eliminating remanent magnetization to treat the patient B after the treatment of the patient A or when resuming the treatment after the treatment is stopped by the interlock operation during the treatment of the patient A due to a certain problem, it is desirable to eliminate the influence of the remanent magnetization as quickly as possible.

However, in the conventional excitation power supplies for elimination of remanent magnetization that are disclosed in PTL 1 and PTL 2, in order to realize relatively low-cost power supplies, demagnetization is performed by generating a damped oscillation current using an electromagnet and an external capacitor, or demagnetization is performed using a pulse magnetic field from the commercial power supply that is cut by the thyristor. Accordingly, when these are applied to the particle beam therapy system of the present invention, it has not been possible to completely eliminate the influence of remanent magnetization in a short time depending on the state of the remanent magnetization after the completion of demagnetization.

The present invention has been made in view of the above, and it is an object of the present invention to enable proper elimination of the remanent magnetization of the scanning magnet, which is used in a particle beam therapy system, in a short time.

### Solution to Problem

A particle beam therapy system according to the present invention is a particle beam therapy system that irradiates an irradiation target with a particle beam accelerated by an accelerator and scanned by a scanning magnet. A power supply to operate the scanning magnet outputs a pattern current for demagnetizing the scanning magnet.

### Advantageous Effects of Invention

According to the present invention, using a power supply for a scanning magnet that performs scanning of a particle beam, a pattern current is made to flow from the power supply at the time of demagnetization, so that the scanning magnet is demagnetized. Therefore, it is easy to change the current pattern and to appropriately eliminate the remanent magnetization within the magnetic pole in a short time with little unevenness.

### BRIEF DESCRIPTION OF DRAWINGS

FIG. 1 is a block diagram showing a main part of a particle beam therapy system of the present invention.

FIG. 2 is a flow chart showing the operation procedure of the particle beam therapy system.

FIG. 3 is a cross-sectional view showing the scanning magnet of the particle beam therapy system of the present invention.

FIG. 4 is a diagram showing the demagnetization current waveform of a first embodiment of the present invention.

FIG. 5 is a diagram showing the demagnetization current waveform of a second embodiment of the present invention.

FIG. 6 is a diagram showing the demagnetization current waveform of a third embodiment of the present invention.

FIG. 7 is a diagram showing the comparison between the demagnetization performance of the present invention and the demagnetization performance in the related art.

FIG. 8 is a diagram showing the frequency dependence of the typical B-H curve.

### DESCRIPTION OF EMBODIMENTS

### First Embodiment

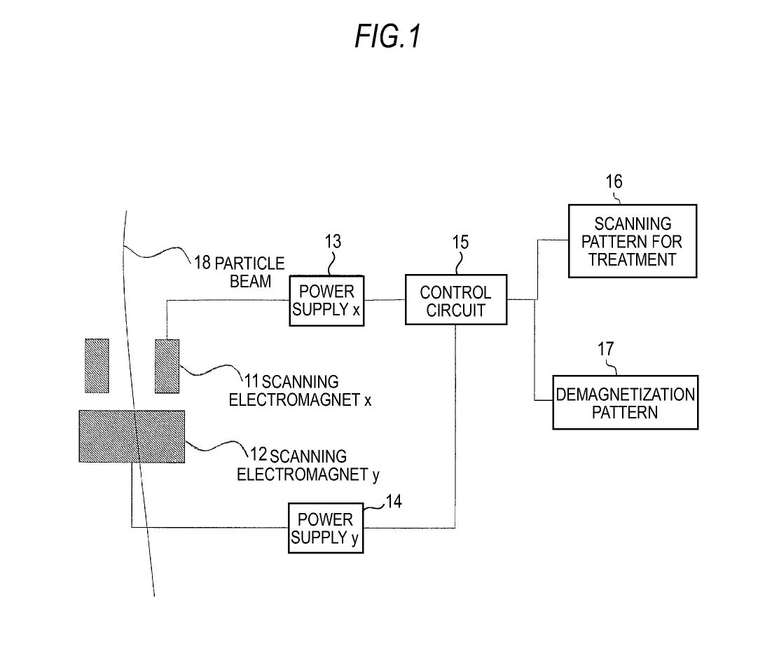

FIG. 1 is a block diagram showing a main part of a particle beam therapy system according to the present invention. In this drawing, a particle beam therapy system includes a scanning magnet (x) 11 that performs scanning in an x-axis direction and a scanning magnet (y) 12 that performs scanning in a y-axis direction. The scanning magnets 11 and 12 are operated by a power supply (x) 13 and a power supply (y) 14 that are connected to a control circuit 15. The control circuit 15 controls the power supplies 13 and 14 in response to commands from a scanning pattern for treatment 16 and a demagnetization pattern 17. During the treatment operation, a particle beam 18 passes between the magnetic poles of the scanning magnets 11 and 12. The control circuit 15 reads the pattern for treatment 16, and this particle beam 18 is scanned by the scanning magnets 11 and 12, which are operated by the pattern currents supplied from the power supplies 13 and 14 according to this pattern, and is irradiated to the affected area of the patient.

On the other hand, during the demagnetizing operation of scanning magnets, the control circuit 15 reads the demagnetization pattern 17 before the start of treatment and (or) after the completion of treatment, and the scanning magnets 11 and 12 are demagnetized by the pattern currents supplied from the power supplies 13 and 14 according to this pattern.

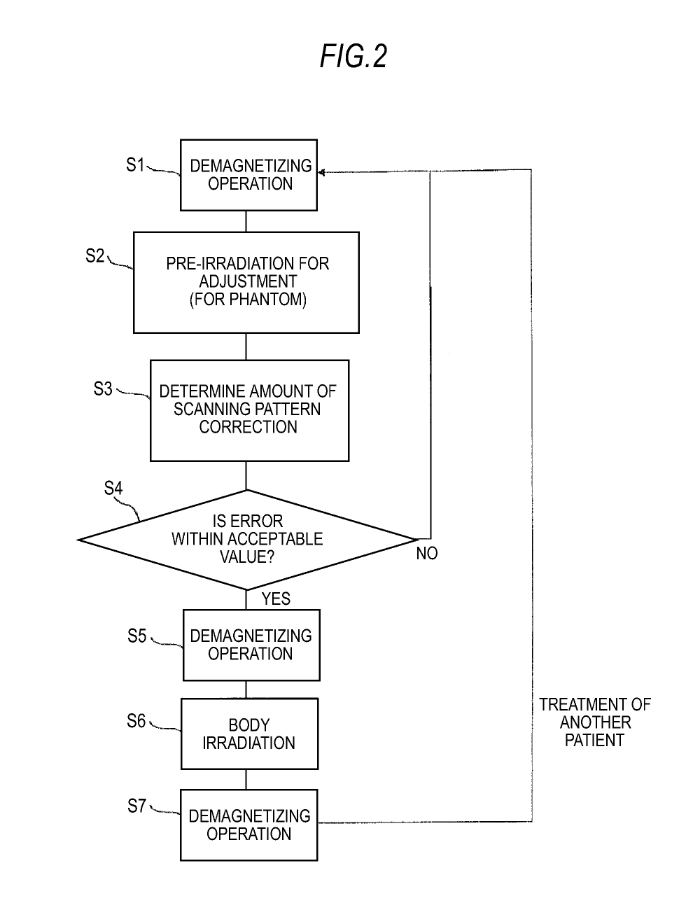

The treatment operation and the demagnetizing operation described above are performed as in the flow chart shown in FIG. 2, for example. A demagnetizing operation is performed before the start of the treatment operation to treat a patient (step S1). Then, pre-irradiation for apparatus adjustment is performed on a phantom (step S2). Then, in order to eliminate the influence of hysteresis of the electromagnet, error factors on the control, and the like, the amount of scanning pattern correction is determined by calculation on the basis of the result (step S3). Then, it is determined whether or not the error after correction is within the acceptable value (step S4). If the error after correction exceeds the acceptable value, the process returns to step S1. If the error after correction is within the acceptable value, the demagnetizing operation is performed (step S5), and then body irradiation that is a treatment operation is performed (step S6). In addition, the demagnetizing operation is performed when necessary (step S7), and the process proceeds to treatment for another patient.

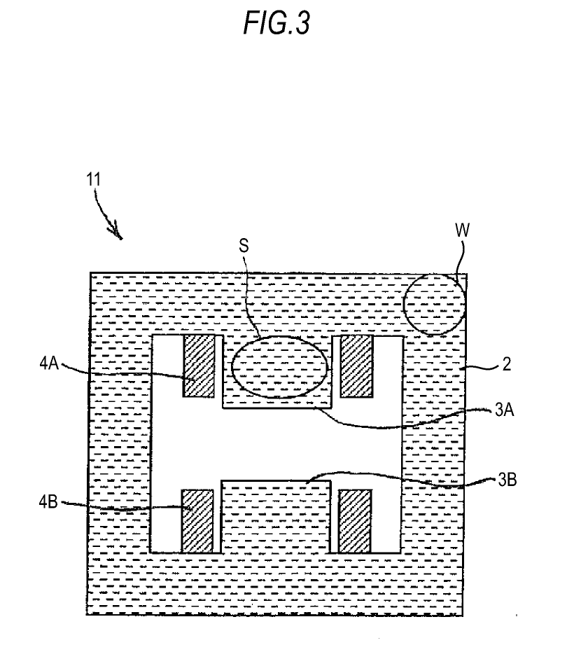

FIG. 3 shows a cross-sectional view of one of the scanning magnets in FIG. 1 (here, the scanning magnet 11). The scanning magnet 11 includes an outer magnetic pole 2 having a mouth-shaped cross-section, a pair of central magnetic poles 3A and 3B provided so as to protrude from the center of the outer magnetic pole and face each other, and coils 4A and 4B wound around the central magnetic poles 3A and 3B. The coils 4A and 4B are connected to the power supply 13 so that current is supplied thereto. The particle beam 18 passes through the opposite region of the central magnetic poles 3A and 3B of the scanning magnet 11 in a vertical direction with respect to the plane of the drawing. At this time, however, the particle beam 18 is deflected on the x axis due to the current flowing through the coils 4A and 4B and is irradiated toward the affected area of the patient.

In order to generate a high-strength magnetic field with low current, iron or electromagnetic steel sheet is generally used as a core material of the scanning magnet 11. For this reason, remanent magnetization remains after current interruption due to the current at the time of particle beam irradiation operation. Remanent magnetization within the magnetic pole has a bias in the magnetic pole depending on the situation of a scan. In the scanning magnet 11 shown in FIG. 3, an S portion has a high magnetic flux density and accordingly, remanent magnetization is likely to remain, and remanent magnetization is relatively difficult to remain in a W portion. Thus, since the strength of the remanent magnetization changes depending on a portion of the magnetic pole, it is necessary to study the demagnetization pattern in order to eliminate the remanent magnetization of each portion of the magnetic pole evenly in each of the coils 4A and 4B wound around the central magnetic poles 3A and 3B.

If remanent magnetization occurs, it is difficult to accurately control a particle beam during the subsequent particle beam irradiation operation. As a result, erroneous irradiation of a particle beam to apart other than the affected area may be caused. In particular, in a scanning device for cancer treatment that is of a type to scan a particle beam using a scanning magnet, remanent magnetization needs to be controlled accurately since the remanent magnetization is a direct cause of the error in irradiation position to the affected area.

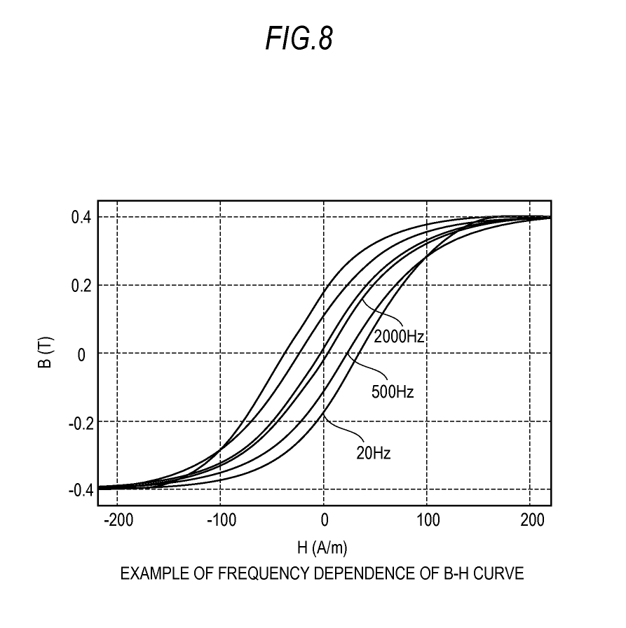

Therefore, it is essential to the particle beam therapy system to demagnetize the scanning magnet 11 time-efficiently and completely evenly using the power supply 13. In the power supply of the scanning magnet, the power supply performance is defined by the maximum voltage and the maximum current. Since the current is a current flowing through the electromagnet, it is an amount having a strong correlation with the maximum magnetic field. In addition, the induced voltage V satisfies V=L×dI/dt (L is the inductance of a magnet, and dI/dt is a current change over time). Therefore, in a certain power supply, it is necessary to reduce the frequency in order to make large current flow when the maximum voltage is regulated, and it is necessary to increase the frequency in order to reduce the current. FIG. 8 shows an example of the B-H curve of the electromagnet. As a general trend, it is known that the hysteresis decreases as the frequency of excitation current increases.

In the present embodiment, therefore, during the demagnetizing operation, current having an amplitude decreasing with time and a frequency increasing with time is used as the current supplied from the power supply 13 to the scanning magnet 11 (referred to as pattern current). This pattern current is generated when the demagnetization pattern 17 capable of setting the current pattern arbitrarily is read into the control circuit 15 and the control circuit 15 controls the power supply 13.

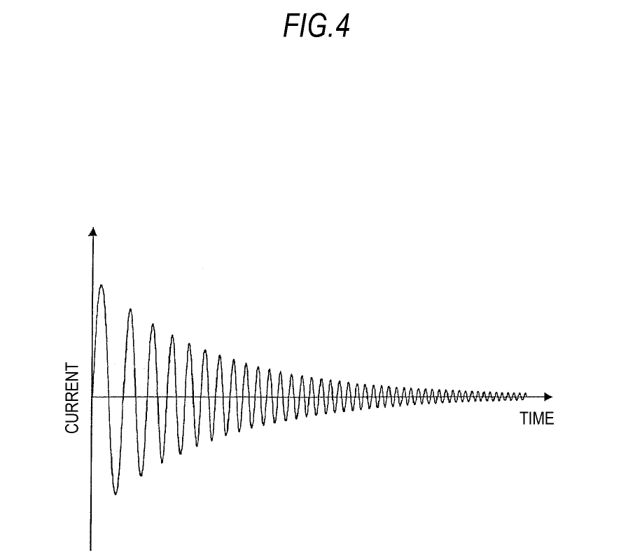

FIG. 4 shows the waveform of this pattern current. This current waveform is an AC waveform whose amplitude decreases with time and frequency increases with time. In FIG. 4, the horizontal axis indicates time t and the vertical axis indicates current amplitude I, and the waveform is expressed as in the following expression.

$$I ( t ) = {I}_{0} sin ( ( {ω}_{0} + α t ) t ) \cdot {e}^{- \frac{t}{τ}} \qquad Expression 1$$

I₀: maximum excitation current, ω₀: initial angular frequency, α: frequency rate of increase, t: time, e: natural logarithm, τ: attenuation time constant

By exciting the magnetic pole with the above-described pattern current flowing from the power supply for scanning 13 to the coils 4A and 4B of the scanning magnet 11, the magnetic flux distribution in the magnetic pole of the scanning magnet 11 changes. Accordingly, both the region S where the remanent magnetization of the scanning magnet 11 is large and the region W where the remanent magnetization of the scanning magnet 11 is small can be evenly and quickly demagnetized by the coils 4A and 4B.

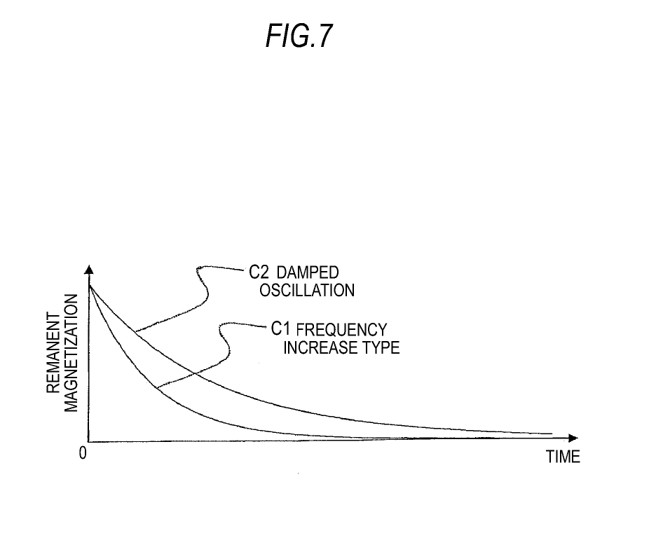

FIG. 7 is a diagram showing the comparison between the conventional demagnetization characteristic C2 using L-C damped oscillation and the frequency increase type demagnetization characteristic C1 due to the pattern current in FIG. 4. As is apparent from the drawing, it can be seen that the demagnetization time in the frequency increase type has been shortened.

Although the above explanation has been given for the scanning magnet 11, the same demagnetizing operation is also required for the scanning magnet 12, and the scanning magnet 12 is demagnetized by the pattern current supplied from the power supply 14 by the setting of the demagnetization pattern 17.

### Second Embodiment

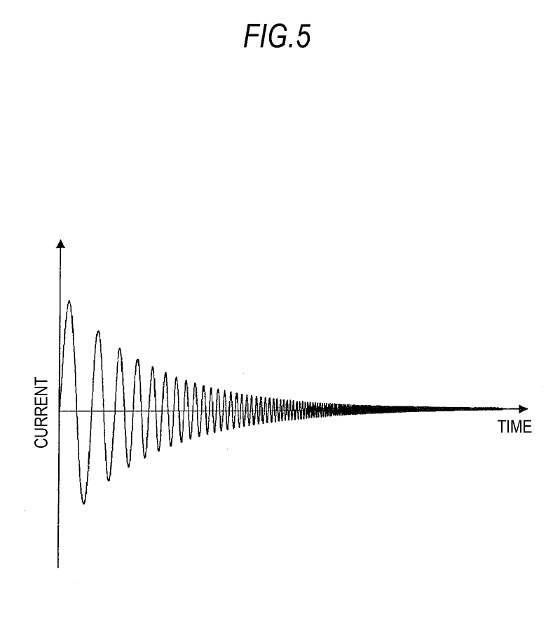

A second embodiment has the same apparatus configuration as the first embodiment. A difference between the first and second embodiments is the waveform of the pattern current for demagnetization that is supplied from the power supply to the scanning magnet. FIG. 5 shows the waveform of the pattern current for demagnetization in a particle beam therapy system according to the second embodiment.

In the present embodiment, the frequency is made to increase with an amplitude decrease so that V=L×dI/dt, which is the induced voltage of the scanning magnet 11, is fixed. This pattern current setting is performed by the demagnetization pattern 17. By performing such an operation, it is possible to perform demagnetization with V=L×dI/dt fixed. Therefore, in addition to the effect of the first embodiment, there is an effect that the performance of the power supplies 13 and 14 can be drawn as much as possible. In FIG. 4, the horizontal axis indicates time t and the vertical axis indicates current amplitude I, and the waveform is expressed as in the following expression.

$$I ( t ) = {I}_{0} sin ( {e}^{\frac{t}{τ}} ) \cdot {e}^{- \frac{t}{τ}} \qquad Expression 2$$

I₀: maximum excitation current, t: time, e: natural logarithm, τ: attenuation time constant

### Third Embodiment

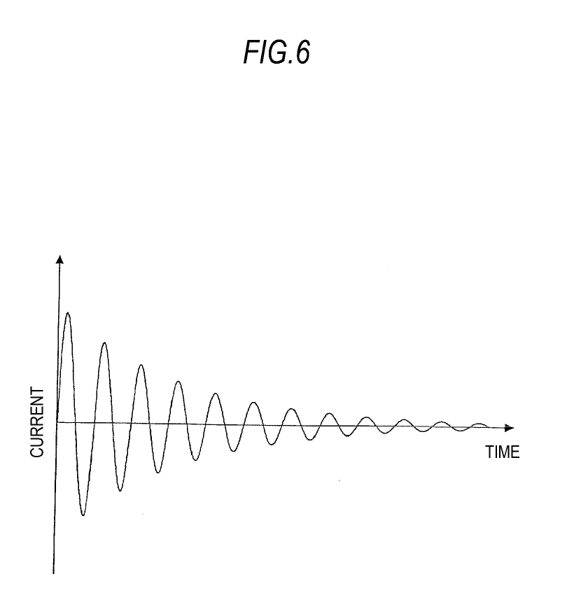

In a third embodiment, as shown in FIG. 6, a current whose frequency is not changed and only amplitude decreases is set as the waveform of the pattern current for demagnetization supplied from the power supply to the scanning magnet. The degree of decrease in the amplitude is set by the demagnetization pattern 17. Effective demagnetization can be performed by performing the setting arbitrarily. In FIG. 6, the horizontal axis indicates time t, and the vertical axis indicates current amplitude I.

As described above, according to the present invention, since demagnetization is performed by supplying the pattern current according to the demagnetization pattern to the scanning magnet from the power supply that scans a particle beam, it is easy to change the current pattern in the power supply for scanning. In addition, since the amplitude or frequency of the demagnetization current can be arbitrarily set, demagnetization can be efficiently performed quickly and with little unevenness. As a result, it is possible to obtain a reliable particle beam therapy system.

### REFERENCE SIGNS LIST

2: outer magnetic pole
3A, 3B: central magnetic pole
4A, 4B: coil
11, 12: scanning magnet
13, 14: power supply
15: control circuit
16: scanning pattern for treatment
17: demagnetization pattern

1-6. (canceled)

7. A particle beam therapy system that irradiates an irradiation target with a particle beam accelerated by an accelerator and scanned by a scanning electromagnet,
wherein a power supply to excite the scanning electromagnet outputs a pattern current for demagnetizing the scanning electromagnet.

8. The particle beam therapy system according to claim 7,
wherein an output current of the power supply is controlled by a control circuit.

9. The particle beam therapy system according to claim 8,
wherein the control circuit reads a demagnetization pattern, and performs control such that a pattern current based on the pattern is output from the power supply.

10. The particle beam therapy system according to claim 7,
wherein the pattern current has a frequency increasing with time.

11. The particle beam therapy system according to claim 7,
wherein the pattern current has an amplitude decreasing with time.

12. The particle beam therapy system according to claim 10,
wherein the pattern current has an amplitude decreasing with time.

13. The particle beam therapy system according to claim 7,
wherein a frequency of the pattern current increases with a decrease in current amplitude so that a generated induced voltage of the scanning electromagnet has a fixed value.
